(19) United States
(12) Patent Application Publication
(10) Pub. No.: US 2008/0108904 A1
(43) Pub. Date: May 8, 2008

(54) IMPLANT FOR SECURING A SENSOR IN A VESSEL

(75) Inventor: Ronald W. Heil, Roseville, MN (US)

Correspondence Address: FAEGRE & BENSON, LLP;BOSTON SCIENTIFIC PATENT DOCK, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/872,107

(22) Filed: Oct. 15, 2007

Related U.S. Application Data
(60) Provisional application No. 60/864,915, filed on Nov. 8, 2006.

(51) Int. Cl. A61B 5/0215

(52) U.S. Cl. 600/486

(57) ABSTRACT

The present invention provides an implantation system for delivering and securing a sensor in a patient's pulmonary artery. In general, the implantation system includes a pulse generator, a catheter, and a sensor coupled to the distal end of the catheter. The sensor is wireless and is adapted to communicate with the pulse generator or an external device.

### CROSS REFERENCE TO RELATED APPLICATION

The present application claims the benefit under 35 U.S.C. § 119 to U.S. Provisional Application No. 60/864,915, filed Nov. 8, 2006, entitled “IMPLANT FOR SECURING A SENSOR IN A VESSEL” which is incorporated herein by reference in its entirety.

### TECHNICAL FIELD

The present invention relates to a system for implanting a medical device, such as a sensor, in a coronary vessel. More specifically, the invention relates to a system for delivering, positioning, and securing a sensor located in the human vasculature.

### BACKGROUND

Medical devices that can be implanted within a patient's body for monitoring one or more physiological parameters and/or to provide therapeutic functions are known. For example, sensors or transducers can be placed in the body for monitoring a variety of properties, such as temperature, blood pressure, strain, fluid flow, chemical properties, electrical properties, and magnetic properties. In addition, implantable medical devices that perform one or more therapeutic functions, such as drug delivery, cardiac pacing, defibrillation, and electrical stimulation are known.

As mentioned above, such implantable medical devices (IMDs) can be configured to measure or sense a number of different physiological parameters in the body. One parameter of particular interest is blood pressure. Implantable pressure sensing modules used in conjunction with cardiac rhythm management (CRM) devices show promise for being able to predict the onset of pulmonary edema in congestive heart failure patients. In addition, certain pressure sensors also may have applications in monitoring and treating hypertension, in automatic CRM device settings optimization, and in rhythm discrimination.

Implanting an IMD generally involves delivering and anchoring the IMD at a desired location in the body. The delivery and anchoring methods and mechanisms can be critical in determining the effectiveness of the IMD. For example, the manner in which the IMD is implanted can be important for patient safety, device placement control, sensor accuracy, long term stability, and physician acceptance and adoption.

There are a number of locations where blood pressure can be measured in a patient's heart. For example, as one skilled in the art will appreciate, pressures measured in the pulmonary artery can be reflective of end diastolic pressures on the left side of the heart. Thus, a need exists for apparatus and/or methods for delivering and securing implantable medical devices within a patient's body.

### SUMMARY

According to an embodiment of the present invention, a system for sensing and communicating a physiological parameter within a cardiac vessel includes: a catheter having a proximal end, a distal end and a lumen extending between the proximal end and the distal end; a guiding element for delivering the distal end of the catheter to a target location in the vessel; a sensor module coupled to the distal end of the catheter, the sensor module including at least one sensing element and a communication element adapted for wireless communication; and a pulse generator.

According to another embodiment of the present invention, a system for sensing and communicating a physiological parameter in the pulmonary artery includes: a catheter having a proximal end, a distal end and a lumen extending between the proximal end and the distal end; a sensor module coupled to the distal end of the catheter, the sensor module including a physiological sensor and a communication element adapted for wireless communication; and at least one fixation member adapted to secure the catheter at a location within the pulmonary artery.

According to yet another embodiment of the present invention a method for sensing and communicating a physiological parameter at a target location within the pulmonary artery includes: coupling a sensor module adapted for wireless communication to a distal end of a catheter having a lumen; inserting a guiding element into the lumen of the catheter; guiding the catheter, including the sensor coupled to its distal end, through a patient's vasculature system to the target location within the pulmonary artery; positioning the sensor at the target location; securing the catheter; and communicating wirelessly with the sensor module.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a schematic view of a system for sensing and communicating a physiological parameter implanted within a patient's heart according to an embodiment of the present invention.

FIG. 2 is a partial sectional view of a system for sensing and communicating a physiological parameter according to an embodiment of the present invention.

FIG. 3A shows a partial sectional view of a system for sensing and communicating a physiological parameter according to an embodiment of the present invention.

FIG. 3B shows a partial sectional view of a system for sensing and communicating a physiological parameter according to another embodiment of the present invention.

FIG. 3C shows a partial sectional view of a system for sensing and communicating a physiological parameter according to another embodiment of the present invention.

FIG. 4A shows a partial sectional view of the system shown in FIG. 3A disposed within the pulmonary artery according to an embodiment of the present invention.

FIG. 4B shows a partial sectional view of the system shown in FIG. 3B disposed within the pulmonary artery according to an embodiment of the present invention.

FIG. 4C shows a partial sectional view of the system shown in FIG. 3C disposed within the pulmonary artery according to an embodiment of the present invention.

FIGS. 5A-5C show schematic views of a system for sensing and communicating a physiological parameter disposed in the pulmonary artery according to various embodiments of the present invention.

FIG. 6A shows a schematic view of a system for sensing and communicating a physiological parameter disposed within the pulmonary artery according to another embodiment of the present invention.

FIG. 6B shows a schematic view of a system for sensing and communicating a physiological parameter according to a further embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

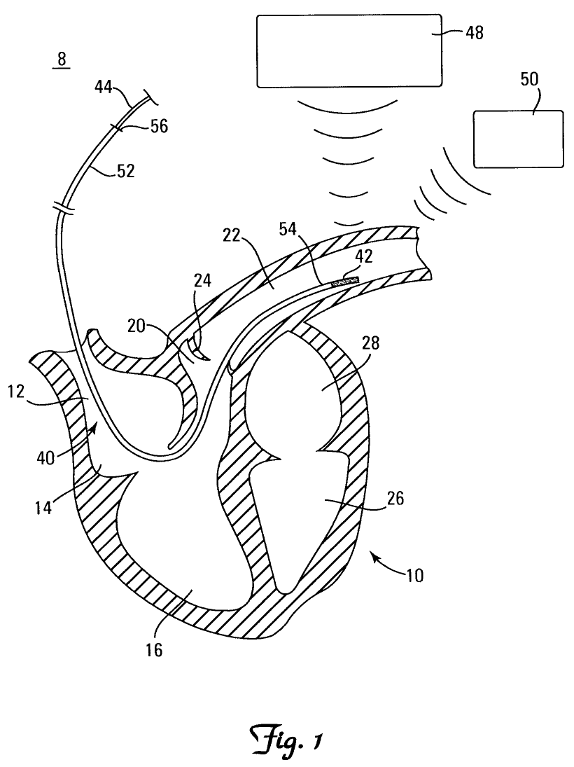

FIG. 1 shows a schematic view of an embodiment of a sensing and communicating system 8 implanted within a patient's heart 10. As shown in FIG. 1, the heart 10 generally includes a superior vena cava 12, a right atrium 14, a right ventricle 16, a ventricular septum 18, a ventricular outflow tract 20, which leads to a pulmonary artery 22 having a pulmonary artery valve 24, a left ventricle 26 and a left atrium 28. In the illustrated embodiment, the system 8 includes an implantation catheter 40, a sensor module 42, and a guiding element 44. The catheter 40 is used in combination with the guiding element 44 to deliver the sensor module 42 to a target location within a patient's heart 10 (e.g. the pulmonary artery). The sensor module 42 is in wireless communication with another implanted device 48, such as a pulse generator adapted to deliver therapy to the heart 10. In an alternative embodiment of the present invention, the sensor module 42 also communicates wirelessly with an external device 50 located outside of the patient's body.

The catheter 40 includes a proximal end 52, a distal end 54, and a lumen 56 extending between the proximal end 52 and the distal end 54. The catheter 40 is formed from a bio-compatible material, such as a flexible bio-compatible polymer, as is generally known in the art. The catheter 40 is sized such that it can effectively couple with the sensor module 42 located at its distal end 54.

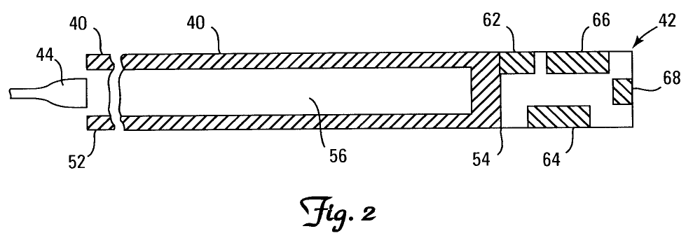

As shown in FIG. 2, the sensor module 42 is coupled to the distal end 54 of the implantation catheter 40. The sensor module 42 may have any shape or size as determined by those of skill in the art. According to one embodiment of the present invention, the sensor module 42 is designed for monitoring blood pressure in the pulmonary artery 22. As will be apparent to those skilled in the art, the sensing and communicating system of the present invention can be adapted to deliver a variety of sensor modules. For example, the sensor module 42 can be used for sensing heart chamber pressure, temperature, blood gas content, strain, fluid flow, chemical properties, electrical properties, magnetic properties, and other physiological parameters. In general, as shown in FIG. 2, the sensor module 42 includes a power source 62, appropriate circuitry 64, a communication element 66, and at least one sensing element 68. The communication element 66 is adapted to transmit to and receive signals from an implanted device 48 and/or an external device 50. The external device 50 typically includes receiving and transmitting elements for communicating with the communication element 66 located in the sensor module 42. In one embodiment according to the present invention, the sensor module 42 is wireless and operates using acoustic, radio, inductive (magnetic) or other telemetries for wireless communication as is known in the art. An exemplary acoustic communication element is found in U.S. Pat. No. 6,486,588 which is herein incorporated by reference. In another embodiment, the communication element 66 is an acoustic communication element that can be activated using an external device 50 adapted to communicate with the communication element 66 located within the sensor module 42. Exemplary devices are described in U.S. Pat. Nos. 6,628,989 and 6,432,050, which are herein incorporated by reference. According to a further embodiment of the present invention, the sensor module 42 includes its own power source so it need not receive power through an electrical lead extending from a device such as the pulse generator 48. Alternatively, the sensor module 42 receives power through an electrical lead provided within the catheter 40.

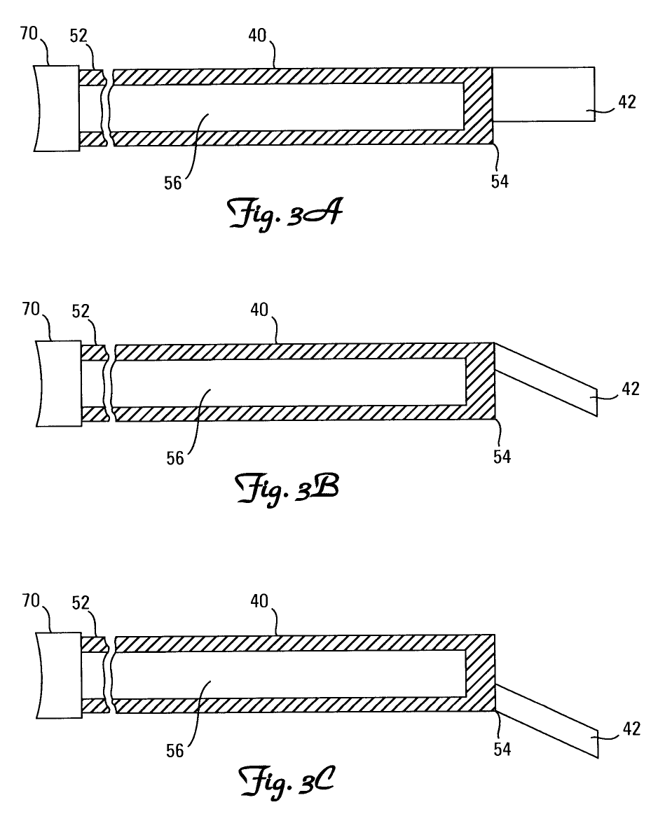

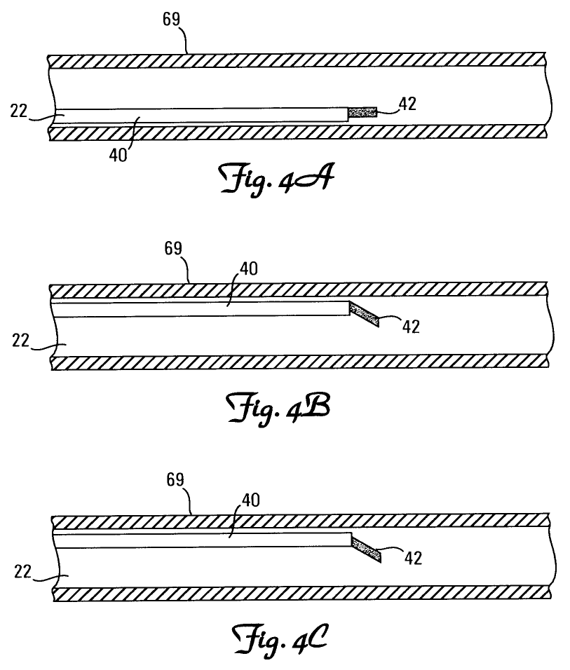

As shown in FIGS. 2, 3A-3C, and 4A-4C the sensor module 42 can couple with the distal end 54 of the implantation catheter 40 in a variety of configurations. According to one embodiment of the present invention, as shown in FIG. 2, a longitudinal axis of the sensor module 42 is in alignment with a longitudinal axis of the catheter 40. In alternate embodiments, as shown in FIGS. 3A-3C and 4A-4C, the longitudinal axis of the sensor module 42 can be offset or angled relative to the longitudinal axis of the catheter 40. The configurations illustrated in FIGS. 3A-3C may enhance sensor performance by allowing the sensor module 42 to be positioned away from an arterial wall 69, as shown in FIGS. 4A-4C.

In a further embodiment of the present invention, as shown in FIGS. 3C and 4C, the sensor module 42 is hingeably attached to the distal end 54 of the catheter 40. In this embodiment, a delivery catheter is used to deliver the system 8 to the target location within the pulmonary artery 22. The sensor module 42 becomes angled relative to the longitudinal axis of the catheter 40 as the delivery catheter is removed and the sensor module 42 is exposed within the pulmonary artery 22.

The sensing and communicating system 8 of the present invention may be left in place in the heart 10 with the catheter 40 adapted to deliver and position the sensor module 42 within the pulmonary artery 22. According to another embodiment of the present invention, as shown in FIGS. 3A-3C, a suture sleeve 70 or its equivalent is provided at the proximal end 52 of the implantation catheter 40 to secure the distal end 54 of the catheter 40 at the target position. In one embodiment, the sensor module 42 is delivered and positioned at a target location within the pulmonary artery 22. In another embodiment, the sensor module 42 is delivered and positioned at a target location in proximity to the bifurcation of the pulmonary artery 22. Once the sensor module 42 is in position at the target location, the catheter 40, according to one embodiment of the present invention, is secured by tethering the catheter 40 to a subcutaneous site in the patient's body using a suture sleeve 70, as shown in FIGS. 3A-3C. By tethering the sensor module 42, the sensor module 42 is prevented from drifting distally beyond the target location in the pulmonary artery 22. Since the sensor module 42 is placed in the direction of blood flow, it will not drift in a proximal direction. The catheter 40, according to an embodiment of the present invention allows the sensor module 42 to be repositioned, as necessary, or retrieved. Alternate means for securing the catheter 40 according to various embodiments of the present invention will be discussed in more detail below.

According to a further embodiment of the present invention, a guiding element 44 is used to guide the distal end 54 of the catheter 40 through the vasculature to a target position in the pulmonary artery 22. The guiding element 44 is inserted into the catheter lumen 56 at the proximal end 52 of the catheter 40. Alternatively, the guiding element 44 may be inserted into a separate lumen formed in the catheter body. The guiding element 44 may be any device known in the art that is appropriately designed to guide and position the implantation catheter 40 into the coronary venous system. The guiding element 44 may be a stylet or may include a single guide wire or multiple guide wires. In one embodiment, as is known in the art, the guide wire or stylet is configured to provide directional bias to the distal end 54 of the implantation catheter 40. Additional guide wires or stylets may be used, as necessary, for adjusting the position of the distal end 54 of the catheter 40. Alternative devices for guiding and positioning the distal end 54 of the catheter 40 may be appropriate. Once the distal end 54 has been positioned at the target location, the guiding element 44 may be removed.

According to yet a further embodiment of the present invention, the catheter 40 may have a predetermined curved shape. A stylet 44 or other guiding element is inserted into the lumen 56 of the catheter 40 to straighten the catheter 40 prior to insertion. The stylet 44 is then used to guide the distal end 54 of the catheter into the pulmonary artery 22. Once the desired position of the distal end 54 of the catheter 40 has been reached, the stylet 44 is removed, allowing the catheter 40 to assume its predetermined curved shape.

According to another embodiment of the present invention, the distal end 54 of the implantation catheter 40 includes a radio-opaque marker. This marker may be used with a fluoroscopic or radiographic device to monitor the location of the catheter 40 within the venous system. In other embodiments, a blind approach or an alternative visualization aid is used in guiding the catheter 40 through the vasculature.

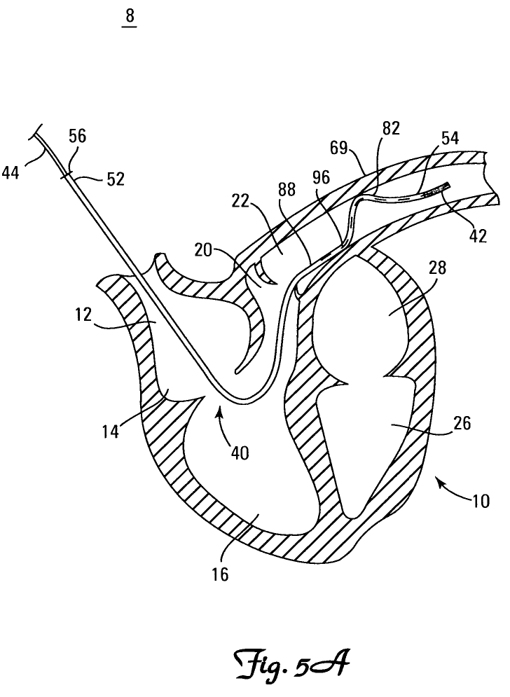

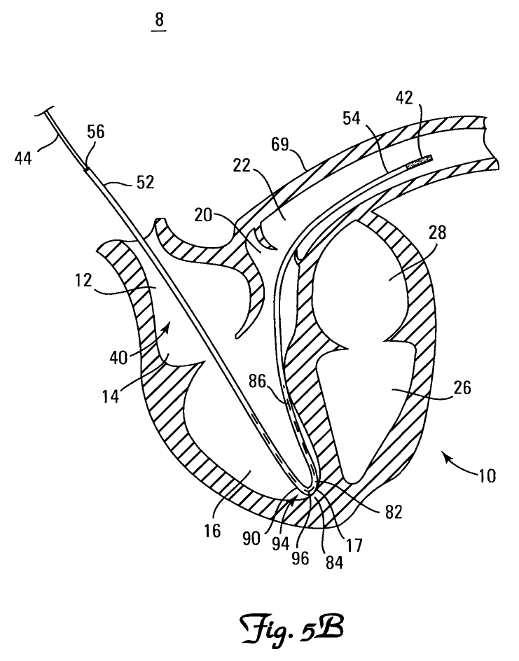

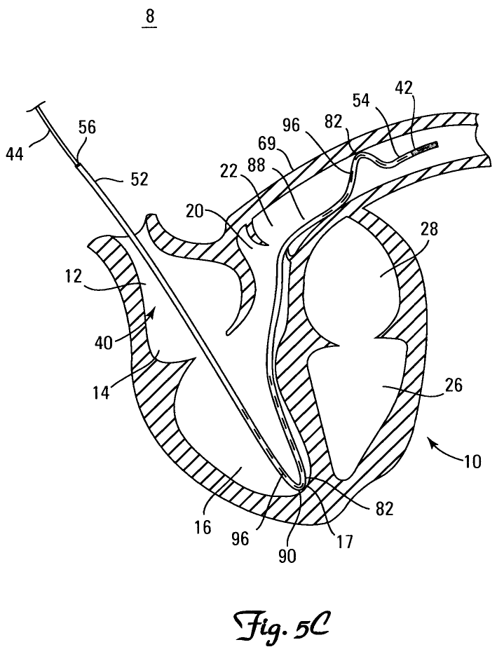

FIGS. 5A-5C show schematic views of the implantation system 8 deployed within a patient's heart 10. As shown in FIGS. 5A-5C, the catheter 40 includes one or more fixation regions 82 and 90. In one embodiment, as shown in FIG. 5A, the fixation region 82 is located at a distal end region 88 of the catheter 40. In an alternative embodiment, as shown in FIG. 5B, the fixation region 90 is located at a middle region 94 of the catheter 40. In yet another embodiment, as shown in FIG. 5C, the catheter 40 includes more than one fixation region 82 and 90. The fixation regions 82 and 90 can be selectively changed between a first or flexible state for implantation of the catheter 40 to the target location (and subsequent removal of the catheter, if desired), and a second or stiffened state for fixation of the distal end 54 within the pulmonary artery 22.

When the fixation region 82 or 90 is in the flexible state, the catheter 40 is sufficiently flexible such that it can be navigated through the coronary venous system using tools and techniques (e.g., guide catheters, guide wires) known in the art. Once delivered to the target position in the pulmonary artery 22, the flow of blood and normal cardiac motion can have the effect of displacing its distal end 54 and, thus, the sensor module 42. Selectively stiffening the fixation region 82 and/or 90 of the catheter 40 (i.e., changing the region to the second or stiffened state) can prevent or significantly impede the spontaneous motion of the distal end 54. In the stiffened state, force is required to remove the distal end 54 from the pulmonary artery 22. As shown in FIG. 5A, when in the stiffened state, the fixation region 82 is constrained by at least one arterial wall 69. This prevents the distal end 54 of the catheter 40 from being dislodged from its target location in the pulmonary artery 22 by the natural motion of the heart 10 or by the flow of blood into the pulmonary artery 22. In another embodiment shown in FIG. 4B, when in the stiffened state, the fixation region 82 is constrained between a bottom portion 84 of the apex 17 and at least one adjacent chamber wall 86 of a patient's heart 10. The stiffened state provides a stable method of securing the sensor module 42 in the pulmonary artery 22.

The fixation regions 82 and 90 can be changed from the flexible state to the stiffened state using one or more fixation members. The fixation member can have any configuration as is known in the art for selectively stiffening a portion or a lead or catheter 40. The fixation member(s), whether inserted into the catheter lumen 56 or provided over the catheter body, is moderately rigid in relation to the implantation catheter 40. The rigid properties of the fixation member(s) prevent proximal or distal movement of the distal end 54 of the catheter 40 relative to the target location.

According to one embodiment, fixation is accomplished by inserting one or more separate internal fixation members 96, indicated by dashed lines in FIGS. 5A-5C, into the lumen 56 of the catheter 40 so as to stiffen a selected region or regions of the catheter 40. The internal fixation member 96 can be a variably flexible wire or internal sheath. Insertion of the fixation member 96 can be accomplished through the use of a guide wire or a stylet or any other suitable technique as is known in the art. If the internal fixation member 96 is provided as an internal sheath, the internal sheath can be inserted over a guide wire provided in the lumen 56 of the catheter 40. The guide wire is removed once the sheath has been secured in the selected fixation region 82. In one embodiment, the fixation member 96 is inserted in a distal region 88. In another embodiment the fixation member 96 is inserted in a middle region 94. In the embodiment shown in FIG. 5C, more than one fixation member 96 may be inserted into the catheter 40. It is appreciated that the location of the fixation member(s) 96 inserted in the implantation catheter 40 is generally determined based on the specific patient anatomy and the target location of the distal end 54 of the catheter 40.

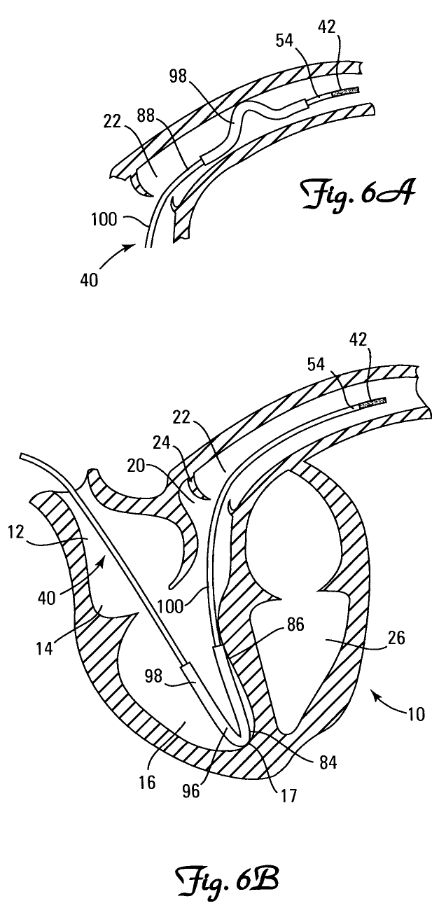

In alternate embodiments of the present invention shown in FIGS. 6A and 6B, the fixation member 98 is an external member (e.g. sheath) adapted to be advanced over the catheter body 100. Fixation is accomplished by sliding one or more separate fixation members 98 over the catheter 40 so as to stiffen a selected region or regions of the catheter 40. In one embodiment, as shown in FIG. 6A, the fixation member 98 is advanced over the catheter body to a distal region 88. In another embodiment, as shown in FIG. 6B, the fixation member 98 is advanced over the catheter body 100 to a middle region 94. In yet another embodiment, more than one fixation member 98 may be provided over the catheter body 100. It is appreciated that the location of the fixation member 98 advanced over the implantation catheter 40 is generally determined based on the specific patient anatomy and the target location of the distal end 54 of the catheter 40.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A system for sensing and communicating a physiological parameter within a cardiac vessel, the system comprising:
a catheter comprising a proximal end, a distal end and a lumen extending between the proximal end and the distal end;
a guiding element for delivering the distal end of the catheter to a target location in the vessel;
a sensor module coupled to the distal end of the catheter, the sensor module comprising electrical circuitry, at least one sensing element, and a communication element adapted for wireless communication; and
a pulse generator.

2. The system according to claim 1, further comprising an external device adapted for wireless communication with the communication element of the sensor module.

3. The system according to claim 1, wherein the pulse generator is adapted for wireless communication with the sensor module.

4. The system according to claim 1, further comprising a suture sleeve located at the proximal end of the catheter.

5. The system according to claim 1, further comprising at least one fixation member adapted to be inserted into the lumen of the catheter for securing the distal end of the catheter at the target location.

6. The system according to claim 5, wherein the fixation member is a variably flexible wire or sheath adapted to be inserted into the lumen of the catheter.

7. The system according to claim 1, further comprising at least one fixation member adapted to be advanced over a body of the catheter for securing the distal end of the catheter at the target location.

8. The system according to claim 6, wherein the fixation member is an external sheath.

9. The system according to claim 1, wherein the target location is a location within the pulmonary artery.

10. The system according to claim 1, wherein the target location is a location in proximity to the bifurcation of the pulmonary artery.

11. The system according to claim 1, wherein the sensing element is a blood pressure sensor.

12. The system according to claim 1, wherein the sensor module extends away from the catheter at an angle toward the center of the vessel.

13. A system for delivering and securing a sensor in the pulmonary artery, the system comprising:
a catheter comprising a proximal end, a distal end and a lumen extending between the proximal end and the distal end;
a sensor module coupled to the distal end of the catheter, the sensor module comprising a physiological sensor and a communication element adapted for wireless communication; and
at least one fixation member adapted to secure the catheter at a location within the pulmonary artery.

14. The system according to claim 13, further comprising at least one device adapted for wireless communication with the communication element of the sensor module.

15. The system according to claim 13, further comprising a guiding element for delivering the distal end of the catheter to a target location within the pulmonary artery.

16. The system according to claim 13, wherein the fixation member is an internal fixation member adapted to be inserted into the lumen of the catheter.

17. The system according to claim 13, wherein the fixation member is an external fixation member adapted to be advanced over a body of the catheter.

18. A method for sensing and communicating a physiological parameter at a target location within the pulmonary artery, comprising:
coupling a sensor module adapted for wireless communication to a distal end of a catheter having a lumen;
inserting a guiding element into the lumen of the catheter;
guiding the catheter, including the sensor coupled to its distal end, through a patient's vasculature system to the target location within the pulmonary artery;
positioning the sensor at the target location;
securing the catheter; and
communicating wirelessly with the sensor module.

19. The method according to claim 18, further comprising changing a fixation region of the catheter from a flexible state to a stiffened state.

20. The method according to claim 18, further comprising inserting one or more fixation members to a fixation region of the catheter.

21. The method according to claim 18, further comprising advancing one or more fixation members over a body of the catheter to a fixation region.

22. The method according to claim 18, further comprising repositioning the sensor module.

23. The method according to claim 18, further comprising retrieving the sensor module.
